(19) United States
(12) Patent Application Publication
(10) Pub. No.: US 2023/0186475 A1
(43) Pub. Date: Jun. 15, 2023

(54) VITAL DATA MEASURING METHOD AND VITAL DATA MEASURING DEVICE

(71) Applicant: PANASONIC INTELLECTUAL PROPERTY MANAGEMENT CO., LTD., Osaka (JP)

(72) Inventors: Masao HAMADA, Fukuoka (JP); Ikuo FUCHIGAMI, Fukuoka (JP); Takeshi YOSHIHARA, Fukuoka (JP)

(73) Assignee: PANASONIC INTELLECTUAL PROPERTY MANAGEMENT CO., LTD., Osaka (JP)

(21) Appl. No.: 17/926,068

(22) PCT Filed: May 24, 2021
(86) PCT No.: PCT/JP2021/019644

§ 371 Date: Nov. 17, 2022

(30) Foreign Application Priority Data
May 25, 2020 (JP) 2020-090694

(51) Int. Cl. G06T 7/00; G06T 7/20; G06T 7/70; G06V 40/20; A61B 5/11

(52) U.S. Cl. CPC G06T 7/0016; A61B 5/1128; G06T 7/20; G06T 7/70; G06V 40/20; G06T 2207/30201; G06T 2207/30221

(57) ABSTRACT

A vital data measuring method includes: acquiring image data of a subject to be measured, wherein the image data are captured by a camera; analyzing the motion of the subject on the basis of the image data of the subject; starting measurement on vital data of the subject using a predetermined range of the image data of the subject in response to the detection of a certain pose of the subject based on an analysis; and outputting measurement results of the vital data of the subject.

### TECHNICAL FIELD

The present disclosure relates to a vital data measurement method and a vital data measurement device.

### BACKGROUND ART

Patent Literature 1 discloses a biological information estimation device that extracts signals (pixel values) in a predetermined range of image data obtained by imaging a person, and outputs to each filter unit, based on different filter coefficients, each signal corresponding to each different coefficient among the extracted signals in the predetermined range. The biological information estimation device estimates, based on an output signal for at least one cycle of the each filter unit and an input signal of the image data (frame) corresponding to the output signal for at least one cycle, a pulse value of a person by an estimation module unit corresponding to the each filter unit, and selects and outputs, according to the output signal of the each filter unit, one of a plurality of pulse values estimated by a plurality of estimation module units. Accordingly, a pulse rate of a user can be estimated in a non-contact manner.

### CITATION LIST

### Patent Literature

Patent Literature 1: Japanese Patent No. 6323809

### SUMMARY OF INVENTION

### Technical Problem

In Patent Literature 1, for example, estimating biological information (for example, pulse rate) of a player of a sport such as a competition is not taken into consideration. For example, when an image of a predetermined range (for example, a skin color region of a face) of the player in the competition is captured by a camera, the following problems may occur. Specifically, there is noise (for example, a fluctuation component) generated by a movement of the player in the competition. Since such noise is generated by an irregular movement of the player or his/her surroundings, measurement accuracy of vital data (for example, pulse rate) of the player deteriorates, thereby making it difficult to stabilize a measurement result. In particular, unlike indoors where a stable illumination is provided, the measurement accuracy of the vital data (for example, pulse rate) of the player tends to further deteriorate outdoors where there is noise (for example, a fluctuation component) due to ambient light (for example, sunlight, light reflected off a surrounding lawn and irradiated to a face, LED light emitted from signage equipment such as advertisements).

The present disclosure is made in view of the above-mentioned situation in the related art, and provides a vital data measurement method and a vital data measurement device that prevent deterioration in measurement accuracy of vital data of a sportsperson to be measured and improve reliability of a measurement result of the vital data.

### Solution to Problem

The present disclosure provides a vital data measurement method including: acquiring image data of a target person imaged by a camera; analyzing a motion of the target person based on the image data of the target person; starting, in response to detection of a specific pose of the target person based on the analysis, measurement of vital data of the target person using image data in a predetermined range of the target person; and outputting a measurement result of the vital data of the target person.

In addition, the present disclosure provides a vital data measurement device including: an acquisition unit configured to acquire image data of a target person imaged by a camera; a motion analysis unit configured to analyze a motion of the target person based on the image data of the target person; a vital analysis unit configured to start, in response to detection of a specific pose of the target person based on the analysis, measurement of vital data of the target person using image data in a predetermined range of the target person; and an output unit configured to output a measurement result of the vital data of the target person.

### Advantageous Effects of Invention

According to the present disclosure, deterioration in measurement accuracy of vital data of a sportsperson to be measured can be prevented and reliability of a measurement result of the vital data can be improved.

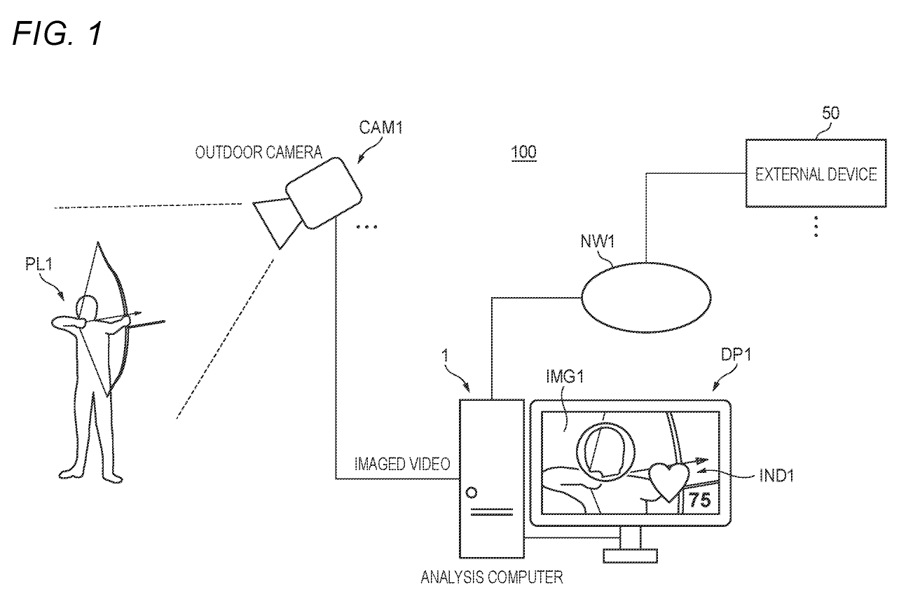

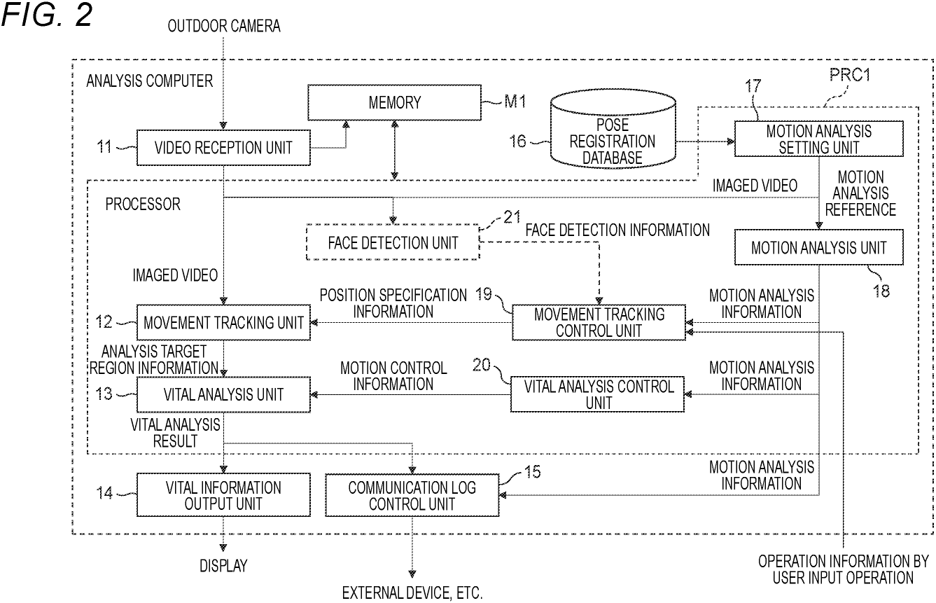

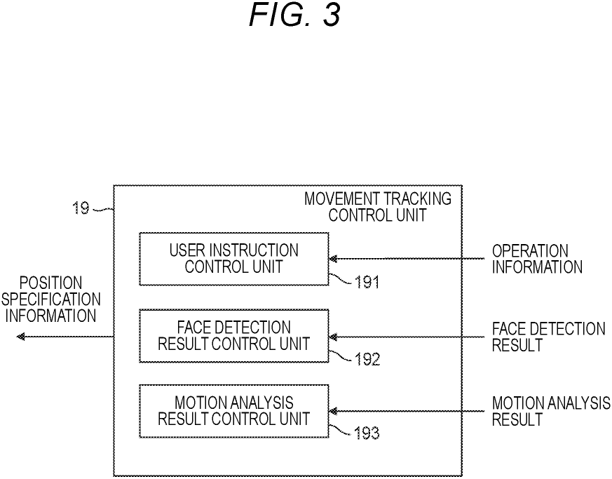

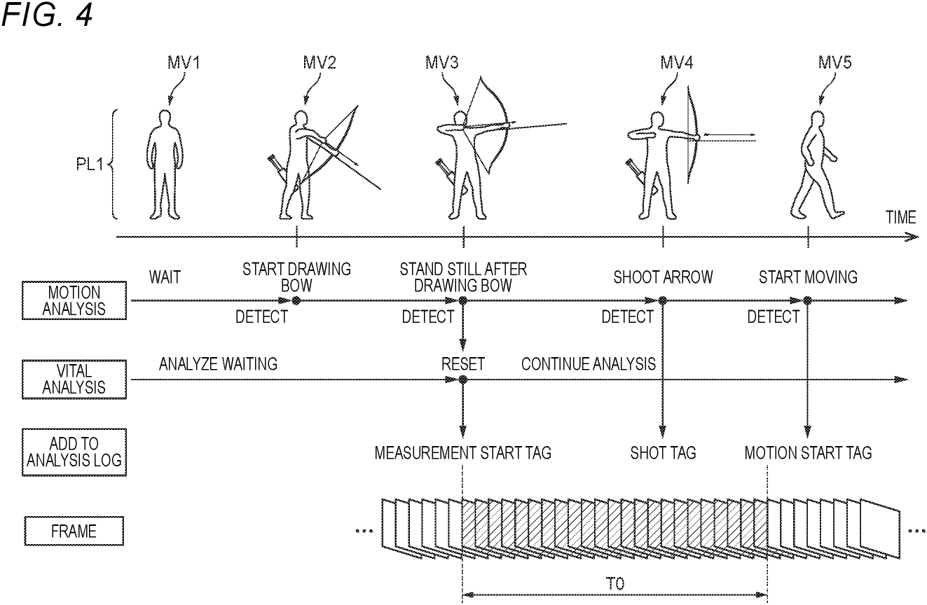

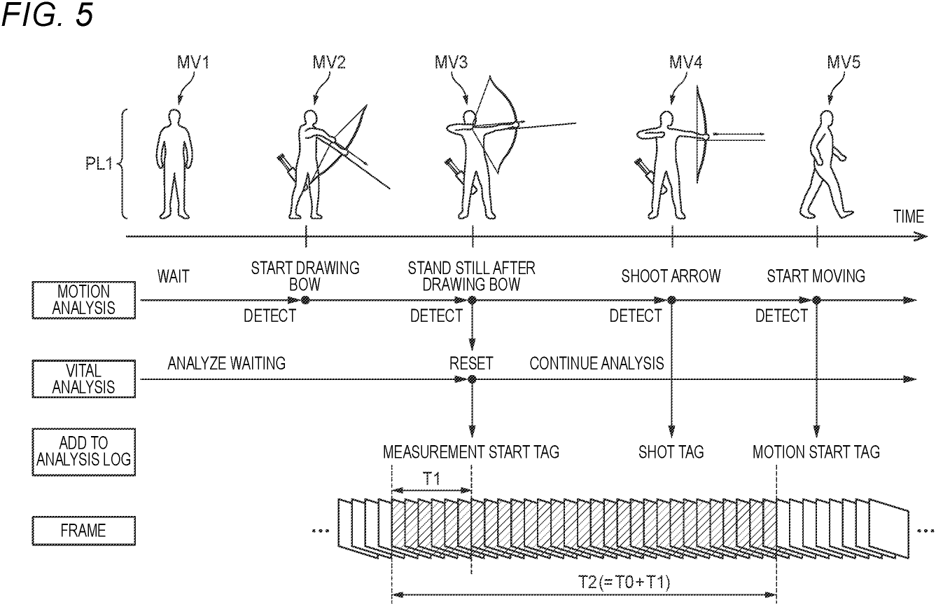

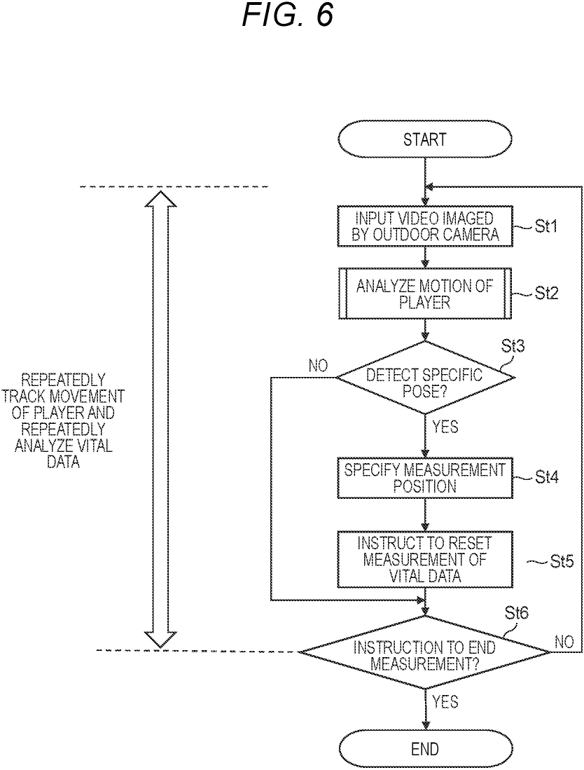

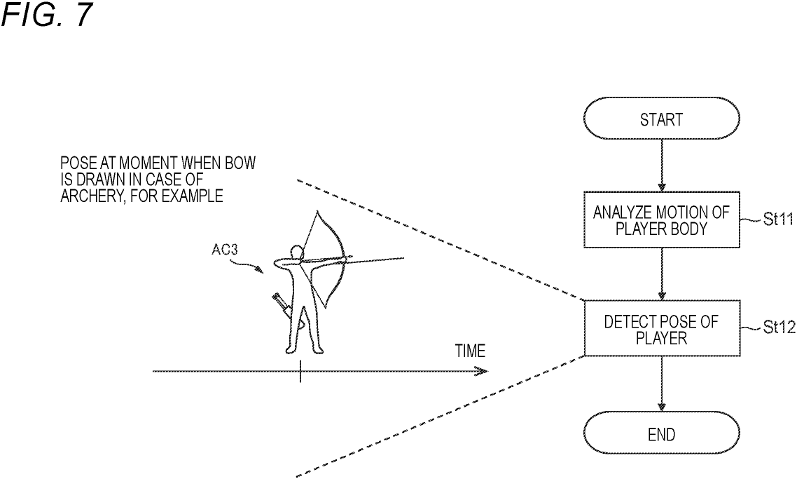

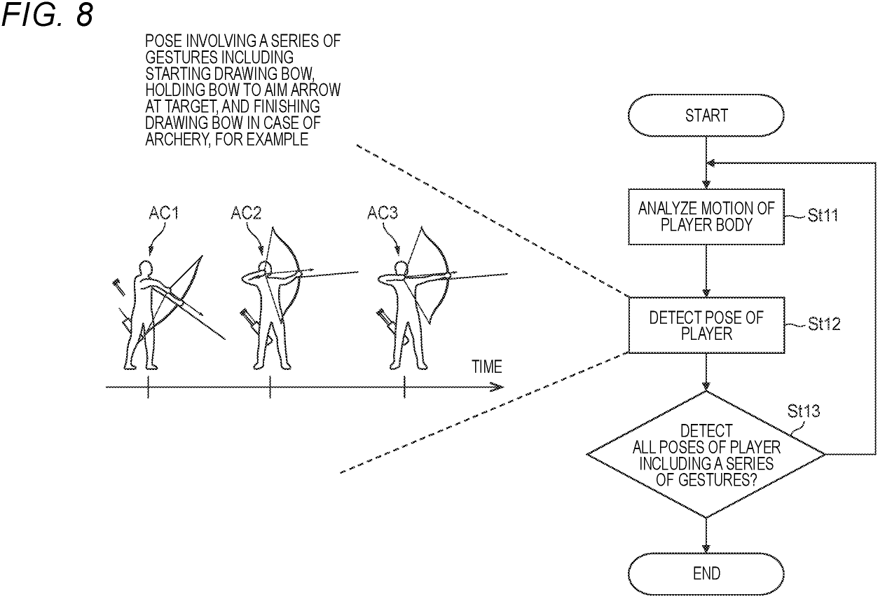

### BRIEF DESCRIPTION OF DRAWINGS

[FIG. 1] FIG. 1 is a schematic diagram showing an example of a configuration of a vital data measurement system according to first embodiment.

[FIG. 2] FIG. 2 is a block diagram showing an example of a hardware configuration of an analysis computer.

[FIG. 3] FIG. 3 is a block diagram showing an example of an internal configuration of a movement tracking control unit in FIG. 2.

[FIG. 4] FIG. 4 is a diagram schematically showing an example of a first operation procedure that defines operation timings of a motion analysis process and a vital analysis process.

[FIG. 5] FIG. 5 is a diagram schematically showing an example of a second operation procedure that defines operation timings of a motion analysis process and a vital analysis process.

[FIG. 6] FIG. 6 is a flowchart showing an example of an operation procedure relating to a motion analysis process and an instruction process for resetting measurement of vital data by an analysis computer according to the first embodiment.

[FIG. 7] FIG. 7 is a flowchart showing an example of a first operation procedure of the motion analysis process in FIG. 6.

[FIG. 8] FIG. 8 is a flowchart showing an example of a second operation procedure of the motion analysis process in FIG. 6.

### DESCRIPTION OF EMBODIMENTS

Hereinafter, embodiments specifically disclosing a vital data measurement device and a vital data measurement method according to the present disclosure will be described in detail with reference to the drawings as appropriate. However, unnecessarily detailed descriptions may be omitted. For example, a detailed description of a well-known matter or repeated descriptions of substantially the same configuration may be omitted. This is to avoid unnecessary redundancy in the following description and to facilitate understanding of those skilled in the art. It should be noted that the accompanying drawings and the following description are provided for a thorough understanding of the present disclosure by those skilled in the art, and are not intended to limit the subject matter recited in the claims.

In the following embodiment, a use case of measuring and outputting, in a non-contact manner, vital data (for example, heart rate variability or average heart rate) of a player (an example of a person to be measured) who is an athlete participating in a sport (for example, archery) such as a competition played outdoors is illustrated and described. However, the sport such as a competition is not limited to archery as long as the sport is played outdoors, and may be, for example, baseball or shooting. In addition, the sport such as a competition may be not limited to being played outdoors, and may be, for example, bowling or curling played indoors.

FIG. 1 is a schematic diagram showing an example of a configuration of a vital data measurement system 100 according to first embodiment. The vital data measurement system 100 includes at least a camera CAM1, an analysis computer 1 connected to a display DP1, and an external device 50. The camera CAM1 and the analysis computer 1 are connected in a wired or wireless manner. The analysis computer 1 and the display DP1 are connected via wire such as a high-definition multimedia interface (HDMI, (registered trademark)) cable. A network NW1 connecting the analysis computer 1 and the external device 50 may be, for example, a wired local area network (LAN), a wireless LAN such as Wi-Fi (registered trademark), or a cellular wireless network such as the fourth generation mobile communication system (4G) or the fifth generation mobile communication system (5G).

The camera CAM1 is installed, for example, for television broadcasting at an archery match venue, is set with an angle of view that can mainly image at least one player PL1 who is an archery athlete, and mainly images the player PL1. The camera CAM1 delivers (transmits) data of an imaged video IMG1 of the player PL1 to the analysis computer 1. It should be noted that although only one camera CAM1 is shown in FIG. 1, it goes without saying that a plurality of cameras CAM1 may be disposed.

The analysis computer 1, which is an example of the vital data measurement device, includes, for example, a personal computer or a high-performance server computer, and receives the data of the imaged video IMG1 delivered by the camera CAM1. The analysis computer 1 analyzes a motion of the player PL1 using the received data of the imaged video IMG1, and measures vital data (for example, heart rate variability or average heart rate) of the player PL1 based on the analysis result. The analysis computer 1 accumulates a measurement result of the vital data of the player PL1, and superimposes the measurement result of the vital data of the player PL1 on the imaged video IMG1 and displays a superimposition result on the display DP1. In addition, the analysis computer 1 may transmit, to the external device 50 via the network NW1, the measurement result of the vital data of the player PL1 or data of the imaged video IMG1 superimposed with an indicator IND1 indicating the measurement result.

The display DP1 is implemented by using, for example, a liquid crystal display (LCD) or an organic electroluminescence (EL) device, and displays data of the imaged video IMG1 superimposed with the vital data of the player PL1 (see FIG. 1). For example, in FIG. 1, the indicator IND1 indicating that the average heart rate is “75” is displayed as the vital data of the player PL1 in a state superimposed on the imaged video IMG1.

The external device 50 may be, for example, a database, or may be a device for a television broadcasting station broadcasting an archery match. The external device 50 may accumulate the measurement result of the vital data of the player PL1, or accumulate the data of the imaged video IMG1 superimposed with the indicator IND1 indicating the measurement result of the vital data of the player PL1 and further deliver the data to another device (not shown).

As described above, in a competition held outdoors such as archery, for example, unlike in an office, it is difficult to obtain a stable illumination, and there is noise (for example, a fluctuation component) due to ambient light (for example, sunlight, light reflected off a surrounding lawn and irradiated to a face, LED light emitted from signage equipment such as advertisements). In addition, there is noise (for example, a fluctuation component) generated by a movement of the player in the competition.

Thus, it can be said that it is a more severe environment for measuring the vital data of the player in a sport played outdoors. For example, in the case of archery, in the video imaged by the camera CAM1, a scene often occurs in which a hand of the player PL1 crosses his/her face until the player PL1 assumes a posture to draw a competition bow. For this reason, a part of the face is hidden by the hand of the player PL1, and reliability (stability) of a measurement result of vital data using data of an imaged image of a face part of the player PL1 is considered to be low.

In many competitions, not limited to archery, there is a mandatory scene (that is, a scene to which a viewer pays particular attention) for which vital data is to be measured, and therefore, it is considered that there is a demand to improve measurement accuracy of the vital data in the mandatory scene. In view of this background, in the first embodiment, the analysis computer 1 specifies, by image analysis, a moment when the player PL1 of the archery assumes a posture to draw, for example, a competition bow, and regards this moment as a timing to start measuring the vital data. Accordingly, it is expected that the measurement accuracy of the vital data in the mandatory scene described above can be improved.

FIG. 2 is a block diagram showing an example of a hardware configuration of the analysis computer 1. The analysis computer 1 includes a memory M1, a video reception unit 11, a movement tracking unit 12, a vital analysis unit 13, a vital information output unit 14, a communication log control unit 15, a pose registration database 16, a motion analysis setting unit 17, a motion analysis unit 18, a movement tracking control unit 19, a vital analysis control unit 20, and a face detection unit 21.

The movement tracking unit 12, the vital analysis unit 13, the motion analysis setting unit 17, the motion analysis unit 18, the movement tracking control unit 19, the vital analysis control unit 20, and the face detection unit 21 are implemented by a processor PRC1. The processor PRC1 is implemented by using, for example, a central processing unit (CPU), a digital signal processor (DSP), or a field programmable gate array (FPGA). That is, the movement tracking unit 12, the vital analysis unit 13, the motion analysis setting unit 17, the motion analysis unit 18, the movement tracking control unit 19, the vital analysis control unit 20, and the face detection unit 21 are functionally constructed in the processor PRC1 by reading and executing a program and data stored in a ROM (see below) of the memory M1 by the processor PRC1.

The memory M1 is implemented by using the random access memory (RAM) and a read only memory (ROM), and temporarily stores a program necessary for executing an operation of the analysis computer 1, as well as data or information generated during the operation. The RAM is, for example, a work memory used during an operation of the processor PRC1. The ROM stores in advance, for example, a program and data for controlling the processor PRC1. It should be noted that the memory M1 may further include a data recording device such as a hard disk drive (HDD) or a solid state drive (SSD) in addition to the RAM and the ROM.

The video reception unit 11 as an example of an acquisition unit is implemented by using a communication interface circuit that controls communication with the camera CAM1, receives the data of the imaged video IMG1 delivered from the camera CAM1 and outputs the data to the processor PRC1, and temporarily stores the data in the memory M1.

The movement tracking unit 12 tracks, based on position specification information sent from the movement tracking control unit 19 and for each frame (imaged image) forming the imaged video IMG1 from the video reception unit 11, a region in a predetermined range (for example, a skin color region of the face of the player PL1) in which vital data in the frame should be analyzed. The movement tracking unit 12 sends information of the region of the predetermined imaging range for each frame obtained by the tracking process to the vital analysis unit 13 as analysis target region information.

The vital analysis unit 13 measures (analyzes), based on motion control information sent from the vital analysis control unit 20 and for each corresponding frame, vital data by using an imaged image of the analysis target region information (for example, the skin color region of the face of the player PL1) for each frame from the movement tracking unit 12. Since a main constituent operation of the vital analysis unit 13 is disclosed in the above-mentioned Patent Literature 1, a detailed description of the contents is omitted. The vital analysis unit 13 sends a measurement result (for example, a result that the average heart rate of the player PL1 is “75”) of the vital data for each frame to the vital information output unit 14 and the communication log control unit 15 as a vital analysis result.

The vital information output unit 14 as an example of an output unit is implemented by using a communication interface circuit that controls communication with the display DP1, and outputs (for example, displays) the vital analysis result from the vital analysis unit 13 on the display DP1. Accordingly, a user of the vital data measurement system 100 can visually and simply obtain the vital data as an indicator indicating how nervous the player PL1 of the archery imaged by the camera CAM1 is while viewing the imaged video.

The communication log control unit 15 is implemented by using a communication interface circuit that controls communication with the network NW1, and transmits data (for example, the vital analysis result) to, for example, the external device 50 connected via the network NW1 and receives an acknowledgment (a log such as Ack) from a transmission destination. In addition, the communication log control unit 15 stores motion analysis information from the motion analysis unit 18 in the memory M1 or a recording device (not shown) as a motion analysis log.

The pose registration database 16 as an example of a database is implemented by using the recording device such as the HDD or the SSD (see above), and accumulates data of a motion analysis reference indicating a specific pose (for example, a pose performed by a player in a mandatory scene to which a viewer pays attention) that differs for each sport such as a competition. The motion analysis reference may be, for example, image data that serves as a correct answer (teacher) for specifying a specific pose, or coordinate information of a possible region on image data when one or more exemplary persons perform a movement same as the specific pose. The one or more exemplary persons may be set for each gender such as male or female, or may be set for each gender and age.

In addition, the pose registration database 16 may accumulate a motion analysis reference indicating a specific pose (see above) that differs for each imaging angle of the camera CAM1 for a sport such as a competition. In addition, the pose registration database 16 may accumulate a motion analysis reference indicating a series of gestures including a specific pose. The series of gestures (details will be described later) includes a specific pose performed by a movement of a body of the player PL1 who is concentrating on the competition, is formed of a plurality of poses (for example, a routine motion including a plurality of poses) in which the movement changes continuously over time as the competition progresses. In addition, the pose registration database 16 may accumulate a motion analysis reference indicating three-dimensional data of a specific pose (see above) mapped independently of the imaging angle, and/or a motion analysis reference indicating a specific pose (see above) that differs for each angle of a camera dedicated to pose detection (not shown) different from the camera CAM1. Further, the pose registration database 16 may accumulate data indicating a result of a learning process of a feature for each specific pose (see above) by machine learning or the like. In this case, the motion analysis unit 18 determines, with reference to the data indicating the result of the learning process of the feature for each specific pose (see above), whether the specific pose (see above) has been detected.

The motion analysis setting unit 17 sets, in the motion analysis unit 18, a motion analysis reference indicating a specific pose suitable for a corresponding sport selected by the user from among the motion analysis references registered in the pose registration database 16. The motion analysis reference is set, for example, when the vital data measurement system 100 is initialized, or when a sport for which vital data is to be measured is changed, but the motion analysis reference may be set at a time other than these times.

The motion analysis unit 18 performs, by using the data of the imaged video IMG1 from the video reception unit 11 and the motion analysis reference set by the motion analysis setting unit 17, image analysis (for example, a skeleton detection process) for each frame (imaged image) forming the imaged video IMG1. Thus, presence or absence of a specific pose of the player PL1 in the frame which is a subject of the image analysis is analyzed. The motion analysis unit 18 sends, to the movement tracking control unit 19, the vital analysis control unit 20, and the communication log control unit 15, respectively, the motion analysis information (for example, pose type information indicating an motion, position information indicating a region of the player PL1 who take the pose in the frame) as a motion analysis result obtained by the motion analysis process. A detailed operation procedure of the motion analysis unit 18 will be described later with reference to FIGS. 7 and 8, respectively.

The movement tracking control unit 19 specifies, based on any one of the motion analysis information from the motion analysis unit 18, operation information by a user input operation, and face detection information from the face detection unit 21, the position specification information indicating a position of a target to be tracked by the movement tracking unit 12 (for example, the skin color region of the face of the player PL1) and sends the position specification information to the movement tracking unit 12. An example of a detailed configuration of the movement tracking control unit 19 will be described with reference to FIG. 3.

FIG. 3 is a block diagram showing an example of an internal configuration of the movement tracking control unit 19 in FIG. 2. The movement tracking control unit 19 includes a user instruction control unit 191, a face detection result control unit 192, and a motion analysis result control unit 193. As described above, the movement tracking control unit 19 is implemented by the processor PRC1.

The user instruction control unit 191 acquires, from an input operation device (for example, a mouse, a keyboard, and a touch panel (not shown)), the operation information obtained by the user operating the input operation device, and sends, to the movement tracking unit 12, the position specification information indicating the position of the target to be tracked (see above) included in the operation information. That is, the face region of the player PL1 for which the vital data is to be measured is specified by a user input operation, and the specified information is input to the movement tracking unit 12 almost as it is as the position specification information.

The face detection result control unit 192 acquires, based on the face detection information from the face detection unit 21, the position specification information indicating the position of the target to be tracked (see above) and sends the position specification information to the movement tracking unit 12. That is, the face region of the player PL1 for which the vital data is to be measured is used as position information of the face region included in the face detection information from the face detection unit 21, and the face detection information is input to the movement tracking unit 12 as the position specification information.

The motion analysis result control unit 193 acquires, based on the motion analysis information from the motion analysis unit 18, the position specification information indicating the position of the target to be tracked (see above) and sends the position specification information to the movement tracking unit 12. That is, the face region of the player PL1 for which the vital data is to be measured is used as position information of the face region in the region of the player PL1 included in the motion analysis information from the motion analysis unit 18, and the position information is input to the movement tracking unit 12 as the position specification information.

The vital analysis control unit 20 generates, based on the motion analysis information from the motion analysis unit 18, the motion control information (for example, a measurement start tag, a shot tag, a motion start tag) that defines an operation timing (for example, operation start, operation stop, operation reset) of the vital analysis unit 13 and sends the motion control information to the vital analysis unit 13. Details of the operation timing of the vital analysis unit 13 will be described later with reference to FIGS. 4 and 5, respectively.

The face detection unit 21 detects, by performing image analysis for each frame (imaged image) forming the imaged video IMG1 based on the data of the imaged video IMG1 from the video reception unit 11, the face region of the player PL1 in the frame. The face detection unit 21 sends the face detection information indicating a position of the face region in a corresponding frame to the movement tracking control unit 19. It should be noted that the face detection unit 21 may be omitted as a component of the analysis computer 1.

Next, details of the operation timing of the vital analysis unit 13 will be described with reference to FIGS. 4 and 5, respectively.

FIG. 4 is a diagram schematically showing an example of a first operation procedure that defines operation timings of a motion analysis process and a vital analysis process. The uppermost stage of FIG. 4 shows a time axis and corresponding types of archery poses of the player PL1. The analysis computer 1 can detect individual movements (poses) of the player PL1 of the archery based on the image analysis by the motion analysis unit 18. Specifically, a pose MV1 in a state in which the player PL1 waits until the other player finishes shooting an arrow, a pose MV2 when the player PL1 starts drawing his/her own competition bow, a pose MV3 in a state in which the player PL1 is standing still after drawing his/her competition bow, a pose MV4 when the player PL1 shoots his/her own competition arrow, and a pose MV5 when the player PL1 starts moving after shooting the his/her own competition arrow are detected respectively.

A mandatory scene in archery (that is, a scene to which a viewer pays particular attention) is, for example, a scene from a state in which the player PL1 is standing still and aiming at a target after drawing the competition bow (see the pose MV3) to a time when the player PL1 shoots the arrow (see the pose MV4) or starts moving after shooting the arrow (see the pose MV5). In a state from the pose MV3 to the pose MV4 or the pose MV5, the face of the player PL1 is not hidden by the hand, and therefore, it is considered that the measurement accuracy of the vital data of the player PL1 does not deteriorate. In order not to deteriorate the measurement accuracy of the vital data of the player PL1, not hiding the face by a tool used in the competition or not moving the face may be considered in addition to not hiding the face of the player PL1 by the hand. It should be noted that the mandatory scene may be not limited to the states described above.

Therefore, in the first operation procedure shown in FIG. 4, when the pose MV3 is detected by the motion analysis unit 18, the vital analysis control unit 20 generates a measurement start tag of the vital data of the player PL1, and sends the measurement start tag to the vital analysis unit 13. In addition, when the pose MV4 is detected by the motion analysis unit 18, the vital analysis control unit 20 generates a shot tag indicating that the player PL1 shoots an arrow, and sends the shot tag to the vital analysis unit 13. Further, when the pose MV5 is detected by the motion analysis unit 18, the vital analysis control unit 20 generates a motion start tag indicating that the player PL1 starts moving after shooting the arrow, and sends the motion start tag to the vital analysis unit 13.

When the vital analysis unit 13 receives the measurement start tag (in other words, an instruction for resetting measurement of the vital data), the vital analysis unit 13 resets a frame of an imaged image accumulated in the memory M1 until the measurement start tag is received, and starts measuring the vital data by using the frame accumulated in the memory M1 after the reset process. Then, when the vital analysis unit 13 receives the shot tag or the motion start tag, the vital analysis unit 13 measures (analyzes) the vital data of the player PL1 by using each frame during time T0 from a time of receiving the measurement start tag to a time of receiving the shot tag or the motion analysis tag. It should be noted that the vital analysis unit 13 may measure (analyze) the vital data of the player PL1 by using the frame accumulated in the memory M1 until the measurement start tag is received.

FIG. 5 is a diagram schematically showing an example of a second operation procedure that defines operation timings of a motion analysis process and a vital analysis process. In the description of FIG. 5, the same elements as those in FIG. 4 are denoted by the same reference numerals, the description thereof will be simplified or omitted, and different contents will be described. A difference between FIG. 4 and FIG. 5 is a start timing of using a frame used for measurement (analysis) of the vital data.

Specifically, in the second operation procedure shown in FIG. 5, when the pose MV3 is detected by the motion analysis unit 18 as in the first operation procedure, the vital analysis control unit 20 generates a measurement start tag of the vital data of the player PL1, and sends the measurement start tag to the vital analysis unit 13. When the vital analysis unit 13 receives the measurement start tag, the vital analysis unit 13 starts using frames of imaged images accumulated in the memory M1 at a time earlier than a time when the measurement start tag is received by a time T1, and starts measuring the vital data. Therefore, in the second operation procedure, the vital analysis unit 13 measures (analyzes) the vital data of the player PL1 by using each frame during a time T2 corresponding to time T1 + time T0 (see FIG. 4).

Next, an operation procedure of the analysis computer 1 of the vital data measurement system 100 according to the first embodiment will be described with reference to FIGS. 6, 7 and 8, respectively.

FIG. 6 is a flowchart showing an example of an operation procedure relating to a motion analysis process and an instruction process for resetting measurement of the vital data by the analysis computer 1 according to the first embodiment. The processes shown in FIG. 6 are mainly executed by the processor PRC1 of the analysis computer 1. It should be noted that although the operation procedure relating to the motion analysis process and the instruction process for resetting the measurement of the vital data by the analysis computer 1 is mainly described in FIG. 6, in addition to these processes, the analysis computer 1 repeatedly executes a movement tracking process of the player PL1 and an analysis process (measurement process) of the vital data.

In FIG. 6, the processor PRC1 acquires (inputs), by the video reception unit 11, the data of the imaged video IMG1 of the player PL1 imaged by the camera CAM1 (St1). The processor PRC1 analyzes, by using the data of the imaged video IMG1 and the motion analysis reference set by the motion analysis setting unit 17, the motion of the player PL1 in the motion analysis unit 18 for each frame (imaged image) forming the imaged video IMG1 (St2). Details of the motion analysis process in step St2 will be described later with reference to FIGS. 7 and 8, respectively.

The processor PRC1 determines, in the motion analysis unit 18, whether a specific pose (see FIGS. 4 or 5) is detected as a result of the motion analysis process in step St2 (St3). When the specific pose is not detected (NO in St3), the process of the processor PRC1 proceeds to step St6.

When the processor PRC1 determines that the specific pose is detected (YES in St3), the processor PRC1 specifies, in the vital analysis control unit 20 and based on any one of the motion analysis information from the motion analysis unit 18, the operation information by the user input operation, and the face detection information from the face detection unit 21, the position specification information indicating a position (an example of a measurement position) of a target to be tracked by the movement tracking unit 12 (for example, the skin color region of the face of the player PL1) (St4). When the specific pose is detected by the motion analysis unit 18, the processor PRC1 generates the measurement start tag of the vital data of the player PL1, and instructs the vital analysis control unit 20 to reset the measurement of the vital data based on the generation of the measurement start tag (St5).

When an instruction to end the measurement of the vital data is input (YES in St6), the process shown in FIG. 6 by the processor PRC1 ends. The instruction to end the measurement of the vital data is input by the processor PRC1 accepting the user input operation, for example.

On the other hand, when the instruction to end the measurement of the vital data is not input (NO in St6), the process of the processor PRC1 returns to step St1, and the processes of steps St1 to St6 are repeated until the instruction to end the measurement of the vital data is input.

FIG. 7 is a flowchart showing an example of a first operation procedure of the motion analysis process in FIG. 6. The processes shown in FIG. 7 are mainly executed by the motion analysis unit 18 of the processor PRC1 of the analysis computer 1. In the first operation procedure, the motion analysis unit 18 detects presence or absence of an instantaneous pose as the mandatory scene in archery (see above).

In FIG. 7, the motion analysis unit 18 analyzes, by using the data of the imaged video IMG1 and the motion analysis reference set by the motion analysis setting unit 17, the motion of the player PL1 for each frame (imaged image) forming the imaged video IMG1 (St11). The motion analysis unit 18 detects the pose of the player PL1 by the motion analysis process in step St11 (St12).

For example, in the case of archery, the player PL1 assumes a posture to shoot the arrow towards the target after drawing the competition bow. A pose AC3 at this time is considered to be the mandatory scene in archery. Therefore, the motion analysis unit 18 detects, as the specific pose, the pose AC3 at a moment when the player PL1 assumes a posture to shoot the arrow towards the target after drawing the competition bow.

As in the archery example described above, the specific pose (or mandatory scene) is not a pose in a relaxed state such as when the player PL1 is resting, and may be selected from among motions during play of the player PL1 who is concentrating on the competition. Accordingly, the measured vital data can be used for analyzing a state of mind of the player PL1 during play (for example, a state of mind in the mandatory scene described above). In addition, the specific pose may be, for example, a pose included in the series of gestures (details will be described later) performed by the player PL1. This pose may be any one of a first pose, a middle pose, and a last pose in the series of gestures. Alternatively, this pose may be a pose with the longest still time of player PL1 in the series of gestures, or a pose in the series of gestures in a case where a region to be analyzed (for example, the skin color region of the face of the player PL1) is maximized when the player PL1 is imaged by the camera CAM1. By determining an appropriate specific pose according to the competition or the player PL1, it is possible to further restrain influence of noise generated by the movement of the player PL1 on the measurement of the vital data.

FIG. 8 is a flowchart showing an example of a second operation procedure of the motion analysis process in FIG. 6. The processes shown in FIG. 8 are mainly executed by the motion analysis unit 18 of the processor PRC1 of the analysis computer 1. In the second operation procedure, the motion analysis unit 18 detects presence or absence of movements during a certain period that are continuously performed by the series of gestures as the mandatory scene in archery (see above).

In FIG. 8, the motion analysis unit 18 analyzes, by using the data of the imaged video IMG1 and the motion analysis reference set by the motion analysis setting unit 17, the motion of the player PL1 for each frame (imaged image) forming the imaged video IMG1 (St11). The motion analysis unit 18 detects the pose of the player PL1 by the motion analysis process in step St11 (St12). The motion analysis unit 18 determines whether all poses of the player PL1 including the series of gestures are detected (St13). When all the poses of the player PL1 including the series of gestures are detected (YES in St13), the process shown in FIG. 8 by the motion analysis unit 18 ends. On the other hand, when all the poses of the player PL1 including the series of gestures are not detected (NO in St13), the processes of steps St11 to St13 are repeated until all the poses of the player PL1 including the series of gestures are detected.

For example, in the case of archery, the player PL1 performs the following series of gestures (specifically, a pose AC1 in a case of starting drawing the competition bow, a pose AC2 in a case of holding the bow to aim the competition arrow at the target, and a pose AC3 in a case of assuming a posture to shoot the arrow towards the target after drawing the competition bow). The series of gestures is considered to be the mandatory scene in archery or a routine motion of the player PL1. Therefore, the motion analysis unit 18 detects, as one set of poses (specific poses), the three poses including the pose AC1 when the player PL1 starts drawing the competition bow, the pose AC2 when the player PL1 holds the bow to aim the competition arrow at the target, and the pose AC3 at a moment when the player PL1 assumes a posture to shoot the arrow towards the target after drawing the competition bow.

Since the vital data is measured from the specific poses after the series of gestures are detected in this manner, it is possible to further restrain the influence of the noise generated by the movement of the player PL1 on the measurement of the vital data. For example, even when the player PL1 accidentally takes a pose of the pose AC3 or takes a pose similar to the pose AC3 in a practice motion (for example, image training of assuming a posture without holding the competition arrow) or the like, the measurement of the vital data is not started. Since the measurement of the vital data is not started at an erroneous timing in this manner, deterioration in measurement accuracy of vital data can be prevented.

As described above, in the vital data measurement system 100 according to the first embodiment, the analysis computer 1 acquires image data (for example, frames of imaged images forming the imaged video IMG1) of a target person imaged by the camera CAM1, and analyzes a motion of the target person based on the image data of the target person. The analysis computer 1 starts, in response to detection of a specific pose (for example, see the pose MV3 when the player PL1 is standing still after drawing the bow) of the target person based on the analysis, non-contact measurement of vital data of the target person using image data in a predetermined range (for example, a face region) of the target person. The analysis computer 1 outputs a measurement result of the vital data of the target person to the display DP1 or the like.

Accordingly, in the vital data measurement system 100, the analysis computer 1 restrains influence of noise generated by, for example, ambient light (for example, sunlight, light reflected off a surrounding lawn and irradiated to a face, LED light emitted from signage equipment such as advertisements) or a movement of the player PL1, and can measure the vital data from an appropriate time for measuring the vital data (for example, a time when a movement of the player PL1 which should be noted is detected). Therefore, the analysis computer 1 can prevent deterioration in measurement accuracy of vital data of a target person of a sport played indoors or outdoors and can improve reliability of a measurement result of the vital data.

In addition, the analysis computer 1 detects a series of gestures of the target person based on the analysis, and starts the measurement of the vital data of the target person in the specific pose included in the series of gestures. Accordingly, for example, when the analysis computer 1 detects a series of gestures (see above) performed by the player PL1 during a competition, measurement of vital data can be started by detecting a specific pose included in the series of gestures, and therefore, by setting a start timing of the measurement of the vital data as a detection time of the specific pose, it is possible to further restrain influence of noise generated by the movement of the player PL1 on the measurement of the vital data.

In addition, the analysis computer 1 detects a second specific pose (for example, see the pose MV4 when the player PL1 shoots the arrow, or the pose MV5 when the player PL1 starts moving after shooting the arrow) of the target person based on the analysis, and accumulates, in association with identification information (for example, a player ID) of the target person, measurement results of the vital data of the target person which are measured from a detection time of the specific pose (see above) to a detection time of the second specific pose. It should be noted that an accumulation destination may be the memory M1 or the external device 50, for example. Accordingly, the analysis computer 1 can measure, by using frames of imaged images during a certain period in the archery, to which the viewer pays attention and which satisfies a condition that the face of the player PL1 necessary for measuring the vital data is not hidden with the hand by a gesture of the player PL1, the vital data of the player PL1 with high precision and store the measurement result.

In addition, the predetermined range is a face region of the target person. For example, in archery, the specific pose is imaged by the camera CAM1 such that the face region of the target person is not hidden by a hand of the target person. Accordingly, since the face is not hidden by the hand of the player PL1 when the player PL1 takes a specific pose, the analysis computer 1 can measure the vital data of the player PL1 with high precision by using a frame of an imaged image imaged when the specific pose is taken.

In addition, the predetermined range is a face region of the target person. For example, in archery, a motion from the specific pose to the second specific pose (see above) is imaged by the camera CAM1 such that the face region of the target person is not hidden by a hand of the target person. Accordingly, since the face is not hidden by the hand of the player PL1 when the player PL1 performs a motion from the specific pose to the second specific pose, the analysis computer 1 can measure the vital data of the player PL1 with high precision by using frames of imaged images imaged when the motion is performed.

In addition, the analysis computer 1 starts the measurement of the vital data of the target person by further using image data in a predetermined range of the target person from a time earlier than a detection time of the specific pose by a predetermined time. Accordingly, in view of a matter that there is a minute time lag between a time when the player PL1 takes the specific pose and a timing of acquiring the frame of the imaged image used for measuring the vital data, the analysis computer 1 can measure the vital data at the moment when the specific pose is taken by using the frame of the imaged image at a time slightly earlier the time when the specific pose is taken.

In addition, the analysis computer 1 detects the specific pose of the target person based on the pose registration database 16 that accumulates a motion analysis reference indicating the specific pose that differs for each sport. Accordingly, since the specific pose that differs for each sport can be accumulated in the pose registration database 16, the analysis computer 1 can improve versatility of measurement of vital data of an athlete of a sport which is not limited to archery.

In addition, the analysis computer 1 detects the specific pose of the target person based on the pose registration database 16 that accumulates a motion analysis reference indicating the specific pose that differs for each sportsperson. Accordingly, since the specific pose that differs for each characteristic of the sportsperson (for example, gender, age, or a combination thereof) can be accumulated in the pose registration database 16, the analysis computer 1 can improve versatility of measurement of vital data by taking into account an appearance characteristic of a player who play the sport.

In addition, the analysis computer 1 detects the specific pose of the target person based on the pose registration database 16 that accumulates a motion analysis reference indicating the specific pose that differs for each imaging angle of the camera CAM1 for a sport. Accordingly, since an appropriate specific pose that differs for each installation angle of the camera CAM1 for imaging the sport can be accumulated in the pose registration database 16, the analysis computer 1 can improve versatility of measurement of vital data of a player regardless of the installation angle of the camera CAM1.

Although the embodiment has been described above with reference to the accompanying drawings, the present disclosure is not limited to such an example. It will be apparent to those skilled in the art that various changes, modifications, substitutions, additions, deletions, and equivalents can be conceived within the scope of the claims, and it should be understood that such changes and the like also belong to the technical scope of the present disclosure. Components in the above-mentioned embodiment may be combined as desired within a range not departing from the spirit of the invention.

For example, the analysis computer 1 starts measuring the vital data when the series of gestures (see FIG. 8) is detected. Thus, golf is given as an example of the sport. For example, when the player PL1 hits a tee shot, the player PL1 must performs a routine of performing a practice swing twice before setting (that is, assuming a posture to put a golf club head behind a ball) and then swinging to hit the shot. A motion analysis reference for the routine is registered in the pose registration database 16 in advance. Therefore, when a series of gestures of player PL1 including performing the practice swing twice and setting (for example, a specific pose is a pose in a case of setting) is detected, the analysis computer 1 starts measuring vital data of the player PL1 at a timing when the set motion, which is the specific pose, is detected. However, when the player PL1 performs a motion (action) to try to lightly set and imagine a direction of a hit ball before performing the routine described above, the analysis computer 1 does not start measuring the vital data. This is because the analysis computer 1 does not detect the motion of the practice swing which is performed twice and should be performed before the set motion of the player PL1.

It should be noted that the present application is based on a Japanese patent application (Japanese Patent Application No. 2020-090694) filed on May 25, 2020, the content of which is incorporated herein by reference.

### INDUSTRIAL APPLICABILITY

The present disclosure is useful as a vital data measurement method and a vital data measurement device that prevent deterioration in measurement accuracy of vital data of a sportsperson to be measured and improve reliability of a measurement result of the vital data.

### REFERENCE SIGNS LIST

1: analysis computer
11: video reception unit
12: movement tracking unit
13: vital analysis unit
14: vital information output unit
15: communication log control unit
16: pose registration database
17: motion analysis setting unit
18: motion analysis unit
19: movement tracking control unit
20: vital analysis control unit
21: face detection unit
50: external device
100: vital data measurement system
191: user instruction control unit
192: face detection result control unit
193: motion analysis result control unit
CAM1: camera
DP1: display
M1: memory
PRC1: processor

1. A vital data measurement method comprising:
acquiring image data of a target person imaged by a camera;
analyzing a motion of the target person based on the image data of the target person;
starting, in response to detection of a specific pose of the target person based on the analysis, measurement of vital data of the target person using image data in a predetermined range of the target person; and
outputting a measurement result of the vital data of the target person.

2. The vital data measurement method according to claim 1, further comprising:
detecting a series of gestures of the target person based on the analysis, and
stating a measurement of the vital data of the target person in the specific pose included in the series of gestures.

3. The vital data measurement method according to claim 1, further comprising:
detecting a second specific pose of the target person based on the analysis; and
accumulating, in association with the target person, measurement results of the vital data of the target person which are measured from a detection time of the specific pose to a detection time of the second specific pose.

4. The vital data measurement method according to claim 1, wherein
the predetermined range is a face region of the target person, and
the specific pose is imaged by the camera such that the face region of the target person is not hidden by a hand of the target person.

5. The vital data measurement method according to claim 3, wherein
the predetermined range is a face region of the target person, and
a motion from the specific pose to the second specific pose is imaged by the camera such that the face region of the target person is not hidden by a hand of the target person.

6. The vital data measurement method according to claim 1, wherein
the measurement of the vital data of the target person is started by further using image data in a predetermined range of the target person from a time earlier than a detection time of the specific pose by a predetermined time.

7. The vital data measurement method according to claim 1, wherein
the specific pose of the target person is detected based on a database that accumulates a motion analysis reference indicating the specific pose that differs for each sport.

8. The vital data measurement method according to claim 1, wherein
the specific pose of the target person is detected based on a database that accumulates a motion analysis reference indicating the specific pose that differs for each sportsperson.

9. The vital data measurement method according to claim 1, wherein
the specific pose of the target person is detected based on a database that accumulates a motion analysis reference indicating the specific pose that differs for each imaging angle of the camera for a sport.

10. A vital data measurement device comprising:
an acquisition unit configured to acquire image data of a target person imaged by a camera;
a motion analysis unit configured to analyze a motion of the target person based on the image data of the target person;
a vital analysis unit configured to start, in response to detection of a specific pose of the target person based on the analysis, measurement of vital data of the target person using image data in a predetermined range of the target person; and
an output unit configured to output a measurement result of the vital data of the target person.
